(19) United States
(12) Patent Application Publication
(10) Pub. No.: US 2025/0302642 A1
(43) Pub. Date: Oct. 2, 2025

(54) Breast Prosthesis with Realistic Look and Feel

(71) Applicant: American Breast Care, LP, Marietta, GA (US)

(72) Inventors: Robert Halley, Atlanta, GA (US); Amanda Wollnick, Doraville, GA (US)

(21) Appl. No.: 19/088,537

(22) Filed: Mar. 24, 2025

Related U.S. Application Data
(60) Provisional application No. 63/573,011, filed on Apr. 2, 2024.

(51) Int. Cl. A61F 2/52; A61F 2/50

(52) U.S. Cl. CPC A61F 2/52; A61F 2/5044; A61F 2002/526

(57) ABSTRACT

A breast prosthesis includes a cured silicone rubber skin layer having a shape corresponding to a natural breast and defining a cavity therein. A cured silicone substance is disposed in the cavity. In a method of making a breast prosthesis, a mold that defines a breast-shaped cavity therein is generated. A silicone rubber skin material is applied to the cavity. The silicone rubber skin material in the cavity is cured to form a skin layer. A low-oil silicone gel or a no-oil silicone gel is injected into the cavity inside of the skin layer. The low-oil silicone gel or a no-oil silicone gel is cured so as to generate the breast prosthesis. In another method of making, a barrier layer is applied to the skin layer and a silicone oil is injected into the cavity inside of the barrier layer prior to curing.

### CROSS-REFERENCE TO RELATED APPLICATION(S)

This application claims the benefit of U.S. Provisional Patent Application Ser. No. 63/573,011, filed Apr. 2, 2024, the entirety of which is hereby incorporated herein by reference.

### BACKGROUND OF THE INVENTION

### 1. Field of the Invention

The present invention relates to breast prostheses and, more specifically, to a breast prosthesis having a realistic look and feel.

### 2. Description of the Related Art

There are two main types of breast prostheses currently on the market:
Silicone rubber-based breast prostheses (SRBP); and
Silicone gel-based prostheses. (SGBP).

SRBPs are formed from 100% silicone rubber. The rubber can be solid or can be fabricated as a chemical or mechanical foam. The advantages of SRBP are their realistic appearance, their ability to replicate any required shape and their durability. The disadvantage is that their feel and movement do not closely match the movement of real breast tissue.

SRBPs are produced by creating a mold based on scans or measurements of an individual patient or by creating a mold based on a standard shape across a size range. Into this mold, a skin of silicone rubber is applied. The silicone skin material can be applied by brush, spray or other typical coating techniques. After the skin silicone cures, a silicone rubber is injected as fill for the prosthesis. The silicone rubber fill can be solid rubber, or it can be a chemical foam. The fill can also be created as a mechanical foam by adding spheres (other shapes are also feasible) into the mold, injecting silicone, allowing the silicone to cure and then removing the spheres. The realistic look of the prosthesis is achieved in a painting step. Additionally, since the silicone is applied as a liquid and cured at room temperature, many different shapes can readily be achieved. The application of a rubber skin also allows for details such as veins, freckles, moles, and tattoos to be added.

SGBPs are formed from silicone gel encased in a polyurethane film bag. The gel can be modified to have reduced density. The advantages of SGBPs are that they have lifelike movement and feel. The disadvantages are that they tend to be heavier than SRBPs and they have an appearance may correspond closely to a natural breast.

SGBPs are also produced by creating a mold based on scans or measurements of an individual patient or by creating a mold based on a standard shape across a size range. A bag or envelope of a barrier film matched to the mold must be created. The barrier film is often a polyurethane film. The barrier is necessary to contain the silicone oil used in most traditional silicone gel formulations. Silicone gels also are very tacky and prone to tearing. Adding a barrier layer blocks the tacky feel and improves the prosthesis durability. The envelope is filled with silicone gel and the prosthesis is cured with heat. Sturdier molds and heat are required to cure the prosthesis into the proper shape. The construction of the bag as well as the more stringent mold requirements can limit the range of achievable shapes. A barrier envelope is required also because the oil in a traditional silicone gel will migrate through a silicone skin and bleed onto the patient and the patient's clothes when traditional silicone gels are encased in silicone skin.

Therefore, there is a need for a breast prosthesis that has the appearance and versatility of a rubber-based prosthesis combined with movement and feel of gel-based prostheses.

### SUMMARY OF THE INVENTION

The disadvantages of the prior art are overcome by the present invention which, in one aspect, is a breast prosthesis that includes a cured silicone rubber skin layer having a shape corresponding to a natural breast and defining a cavity therein. A cured silicone substance is disposed in the cavity.

In another aspect, the invention is a method of making a breast prosthesis, in which a breast prosthesis mold that defines a breast-shaped cavity therein is generated. At least one silicone rubber skin material is applied to the cavity. The at least one silicone rubber skin material in the cavity is cured to form a skin layer. A selected one of a low-oil silicone gel or a no-oil silicone gel is injected into the cavity inside of the skin layer. The selected one of a low-oil silicone gel or a no-oil silicone gel is cured, thereby generating the breast prosthesis.

In yet another aspect, the invention is a method of making a breast prosthesis, in which a breast prosthesis mold that defines a breast-shaped cavity therein is generated. At least one silicone rubber skin material is applied to the cavity so as to form a skin layer. A barrier layer is applied to the skin layer. A silicone oil is injected into the cavity inside of the barrier layer. The silicone oil is cured, thereby generating the breast prosthesis.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIGS. 1A-1G are schematic diagrams showing a first method of making a breast prosthesis.

FIGS. 2A-2F are schematic diagrams showing a second method of making a breast prosthesis.

FIG. 3 is a front plan view of a breast prosthesis.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

One embodiment of the invention combines the appearance and versatility of rubber-based custom breast prosthesis with the movement and feel of a gel-based prosthesis. The inventors have discovered two methods of making such a prosthesis can achieve the desired result.

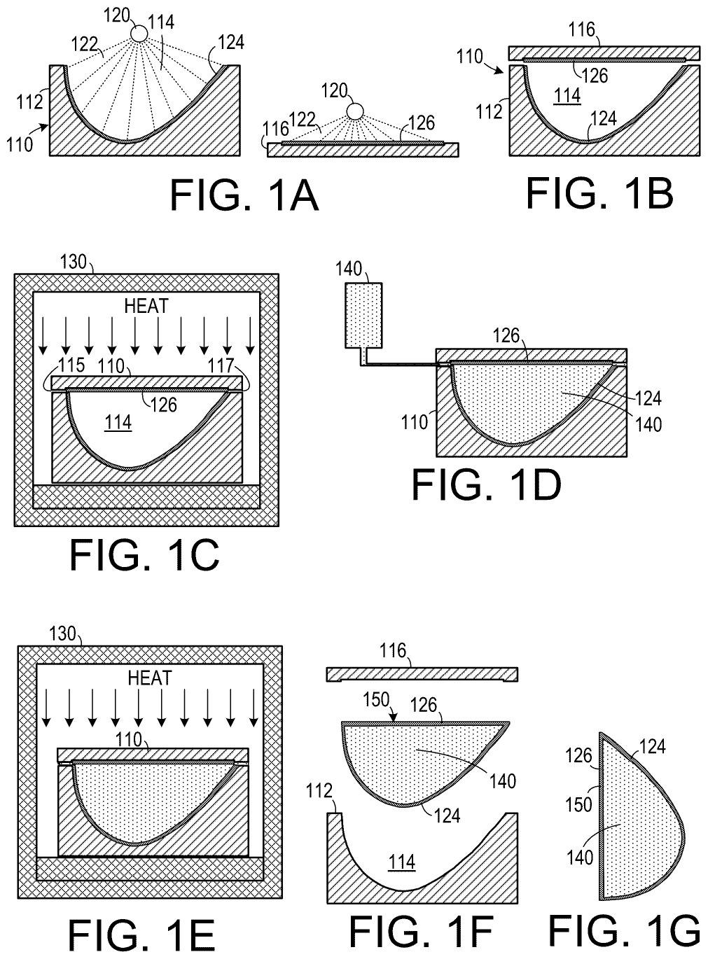

As shown in FIGS. 1A-1G, in a first method (Method 1) of making a breast prosthesis, a mold 110 is generated based either on the scans and measurements of a patient or on a standard shape. The mold 110 can be made from typical tooling board, temperature resistant tooling board, metal or other materials traditionally used for mold making. (Examples include: RAKU® TOOL Modeling Boards (MB) Series). The mold 110 will generally have two parts: a main portion 112 that defines a cavity 114 that has a shape that is complementary to the front of a natural breast and a back plate 116 that has a shape that matches the contours and shape of the user's chest (which can be a surgery site).

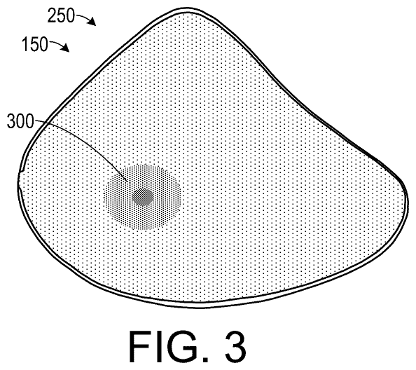

A silicone rubber skin 124 is applied on the cavity 114 and the back plate 116 of the mold 110. The silicone skin 124 material can be applied by a brush, a spray skin material 122 from a sprayer 120 or other known coating techniques. (The materials for silicone skin 124 can include, for example: Smooth-On Ecoflex™ Series, Dragon Skin™ Series). The silicone skin 122 material can be modified with pigments and other fillers to make a more realistic appearance. A nipple 300 (as shown in FIG. 3) and areola feature can be painted on the surface of the cavity first before the skin layer(s) 124 are painted. Multiple skin layers can be painted. A thicker layer 126 of silicone can be used (on the back plate 116 for example) to reinforce the shape of the prosthesis 150. When the final layer of skin is applied on both the back plate and in the cavity, the back plate is placed on the cavity to close the mold, and the final skin layers 124 are allowed to cure. Curing can be accomplished at room temperature when a catalyst has been added to the skin material 122 or it can be accomplished by heating in an oven 130. This seals the periphery of the prosthesis 150. An inlet 115 is allowed to remain for injecting fill, and a vent 117 or multiple vents may also be kept open.

A low-oil or no-oil silicone gel 140 is then injected into the mold 110. Examples of low or no oil silicone gels are presented, for example, in U.S. Pat. No. 5,741,877 (which is incorporated herein by reference), which discloses a silicone gel-like material having a reduced content of extractable silicone fluid. The gel 140 may be modified with pigments, density reducing additives or other additives. (Examples of such additives include pigments from Factor 2 and Fuse FX, flocking powders from Factor 2, microspheres from Expancel, and Phase change materials from Outlast technologies). The prosthesis can be cured at room temperature or at elevated temperature in an oven 130 as dictated by the gel and mold material.

The resulting prosthesis 150 will have the appearance and shape of a SRBP as created from the silicone skin 124 and 126, but it will have the movement of a SGBP made possible by the low-oil gel 140 used in its production. Furthermore, no polyurethane barrier will be required because the low-oil gel 140 does not have the propensity to bleed oil through the skin layer as existing gels have a tendency to do.

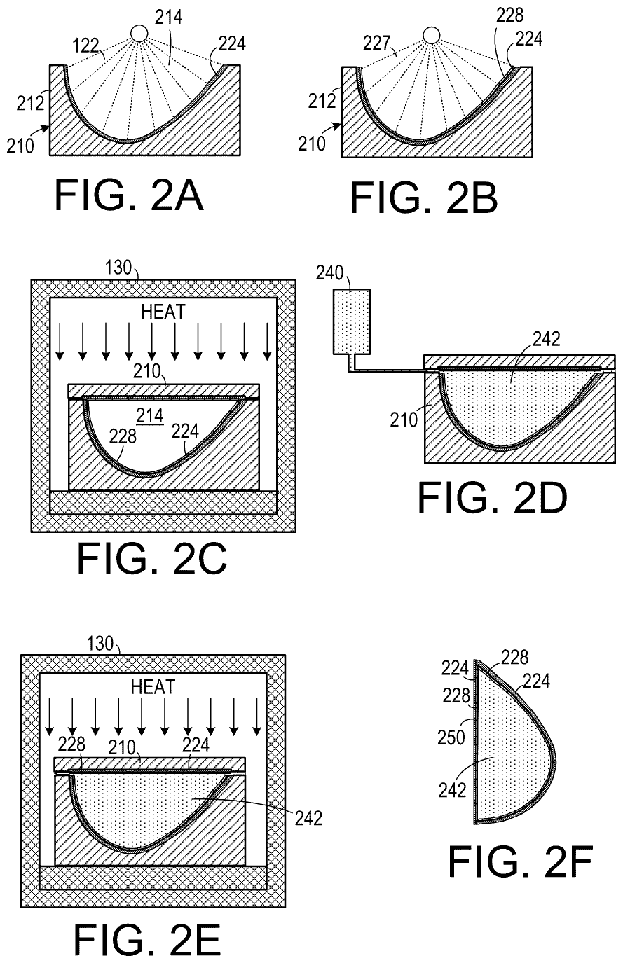

As shown in FIGS. 2A-2F, in a second method (Method 2) of making a breast prosthesis 250, a mold 210 is created based either on the scans and measurements of a patient or on a standard shape. The mold 210 can be made from typical tooling board, temperature resistant tooling board, metal or other materials traditionally used for mold making. (Examples include: RAKU® TOOL Modeling Boards (MB) Series.) The mold 210 will generally have two parts: (1) a cavity 214 which has a shape complementary to that of a natural breast; and (2) a back plate which is shaped to match the contours and shape of the user's chest (usually a surgery site).

A silicone rubber skin 224 is applied to the mold 210 inside the cavity 214 and back plate of the mold 210. The silicone skin material 122 can be applied by brush, spray or other typical coating techniques. (The materials for silicone skin can include, for example: Smooth-On Ecoflex™ Series, Dragon Skin™ Series). The silicone skin material 122 can be modified with pigments and other fillers to make a more realistic appearance. A nipple 300 and areola feature (as shown in FIG. 3) can be painted on the surface of the cavity 214 first before the skin layers 224 are painted. Multiple skin layers can be painted. A thicker layer of silicone can be used (on the back plate for example) to reinforce the shape of the prosthesis.

A barrier layer 228 is applied to the skin layer 224 being constructed on both the cavity 214 and back plate of the mold. It can be applied between two layers of silicone skin, but doing so is not necessary in every embodiment. The barrier layer 228 can include a polyurethane (PU) film that can be applied by spray paint using a solvent based carrier 227. The PU film can be applied as a skin layer is curing and multiple PU layers can be applied. An adhesion promoter or heat can be used to improve adhesion between the PU film and silicone skin. (Appropriate grades of polyurethane can include SC-89-5 from BJBMaterials. Suitable adhesion promoters can include platinum catalysts, silanes, Poly dihydrogen siloxane and Titanium containing compounds.) The barrier layer 228 can also be a modified silicone rubber layer. Silicone rubber modified with fluoride or phenyl groups have been shown to reduce oil bleed through silicone skins. (One example of this is disclosed in U.S. Pat. No. 4,455,691, which is incorporated herein by reference). One or more layers of modified silicone skin can be applied as barrier layers.

When the final layer of skin is applied on both the back plate and in the cavity, the back plate is placed on the cavity to close the mold 210, and the final skin layers 224 are allowed to cure. This seals the periphery of the prosthesis 250. An inlet is allowed to remain for injecting fill, and a vent or multiple vents may also be kept open.

A silicone gel 242 is injected into the mold 210. In this embodiment, a traditional silicone gel can be used as fill for the prosthesis because a barrier layer 228 is in place to block the oil in the gel from migrating through the skin layer 224. The gel may be modified with pigments, density reducing additives or other additives. The prosthesis 250 can be cured at room temperature or at elevated temperature as dictated by the gel and mold material.

The resulting prosthesis 250 will have the appearance and shape of a SRBP as created from the silicone skin 224, but it will have the movement of a SGBP made possible by the silicone gel 242. Furthermore, the polyurethane barrier 228 will limit or eliminate propensity of the gel to bleed oil through the silicone skin.

Methods 1 and 2 are not mutually exclusive. They can be used together to create a hybrid prosthesis.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described herein, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112 (f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A breast prosthesis, comprising:
(a) a cured silicone rubber skin layer having a shape corresponding to a natural breast and defining a cavity therein; and
(b) a cured silicone substance disposed in the cavity.

2. The breast prosthesis of claim 1, wherein the cured silicone substance comprises a selected one of a low-oil silicone gel or a no-oil silicone gel.

3. The breast prosthesis of claim 1, further comprising a barrier layer that is disposed inside the cavity and in contact with the skin layer and wherein the cured silicone substance comprises a cured silicone oil.

4. The breast prosthesis of claim 3, wherein the barrier layer comprises a polyurethane film.

5. The breast prosthesis of claim 3, wherein the barrier layer comprises a silicone rubber modified with a selected one of a fluoride or a phenyl group.

6. The breast prosthesis of claim 1, further comprising an image of a nipple on an outer portion of the skin layer.

7. The breast prosthesis of claim 1, wherein the silicone rubber skin layer has a back portion, configured to be placed against a chest of a user, and an opposite front portion, wherein the back portion has a thickness that is greater than a thickness of the front portion.

8. The breast prosthesis of claim 1, further comprising a plurality of microspheres that have a lower density than the cured silicone substance and that are dispersed in the cured silicone substance.

9. A method of making a breast prosthesis, comprising the steps of:
(a) generating a breast prosthesis mold that defines a breast-shaped cavity therein;
(b) applying at least one silicone rubber skin material to the cavity;
(c) curing the at least one silicone rubber skin material in the cavity to form a skin layer;
(d) injecting a selected one of a low-oil silicone gel or a no-oil silicone gel into the cavity inside of the skin layer; and
(e) curing the selected one of a low-oil silicone gel or a no-oil silicone gel, thereby generating the breast prosthesis.

10. The method of claim 9, further comprising the step of applying an image of a nipple on an outer portion of the skin layer.

11. The method of claim 9, wherein the silicone rubber skin layer has a back portion, configured to be placed against a chest of a user, and an opposite front portion, and further comprising the step of making the back portion so as to have a thickness that is greater than a thickness of the front portion.

12. The breast prosthesis of claim 9, further comprising the step of adding a plurality of microspheres that have a lower density than the cured silicone substance to the selected one of a low-oil silicone gel or a no-oil silicone gel.

13. A method of making a breast prosthesis, comprising the steps of:
(a) generating a breast prosthesis mold that defines a breast-shaped cavity therein;
(b) applying at least one silicone rubber skin material to the cavity so as to form a skin layer;
(c) applying a barrier layer to the skin layer;
(d) injecting a silicone oil into the cavity inside of the barrier layer; and
(e) curing the silicone oil, thereby generating the breast prosthesis.

14. The method of claim 13, wherein the barrier layer comprises a polyurethane film.

15. The breast prosthesis of claim 13, wherein the barrier layer comprises a silicone rubber modified with a selected one of a fluoride or a phenyl group.

16. The breast prosthesis of claim 13, further comprising an image of a nipple on an outer portion of the skin layer.

17. The method of claim 13, wherein the silicone rubber skin layer has a back portion, configured to be placed against a chest of a user, and an opposite front portion, and further comprising the step of making the back portion so as to have a thickness that is greater than a thickness of the front portion.

18. The breast prosthesis of claim 13, further comprising the step of adding a plurality of microspheres that have a lower density than the silicone oil to the silicone oil.
